(19) United States
(12) Patent Application Publication
(10) Pub. No.: US 2018/0055726 A1
(43) Pub. Date: Mar. 1, 2018

(54) PORTABLE ABDOMINAL CARDIOPULMONARY RESUSCITATOR FOR ELECTROCARDIOGRAM MONITORING

(71) Applicants: Lixiang WANG, Beijing (CN); Jing LI, Beijing (CN); Baosheng SU, Beijing (CN)

(72) Inventors: Lixiang WANG, Beijing (CN); Jing LI, Beijing (CN); Baosheng SU, Beijing (CN)

(21) Appl. No.: 15/367,207

(22) Filed: Dec. 2, 2016

(30) Foreign Application Priority Data
Aug. 31, 2016 (CN) 201621032713.7

(51) Int. Cl. A61H 31/00; A61B 5/0402

(52) U.S. Cl. CPC A61H 31/005; A61H 31/007; A61H 2201/5058; A61H 2201/0157; A61B 5/0402

(57) ABSTRACT

The utility model discloses a portable abdominal cardiopulmonary resuscitator for electrocardiogram monitoring, comprising an abdominal pull-push cardiopulmonary resuscitator body provided with a display and a central processing unit, wherein the abdominal pull-push cardiopulmonary resuscitator body is also provided with an electrocardiographic electrode communication module which is connected with the central processing unit; the electrocardiographic electrode communication module is connected with electrocardiographic electrodes in a wireless or wired way; and the display is used for displaying abdominal pull-push information and electrocardiogram monitoring information. According to the utility mode, by adding the electrocardiogram monitoring function on the display and following the abdominal pull-push information, operators can observe the electrocardiographic changes of patients in real time during rescue without raising heads to adjust the pull and push situations of the abdominal cardiopulmonary resuscitator. The abdominal cardiopulmonary resuscitator is conveniently used, is not limited by the application sites, improves the success rate of rescue.

### BACKGROUND OF THE INVENTION

The utility model relates to the field of medical device, in particular to a portable abdominal cardiopulmonary resuscitator for electrocardiogram monitoring.

Methods for cardiopulmonary resuscitation adopted for patients suffering sudden heart arrest includes thoracic cardiopulmonary resuscitation and abdominal cardiopulmonary resuscitation. The thoracic cardiopulmonary resuscitation is carried out by medical staff through giving mouth-to-mouth artificial respiration and pushing the chest with a frequency with two hands, and the abdominal cardiopulmonary resuscitation is mainly carried out by pull-push actions. A utility model, patent No. zI200920160376.3, discloses an induction-controlled type abdominal pull-push cardiopulmonary resuscitator, which achieves goods effects in aspiration and circulation, is safe and sanitary, can be operated by one person, and therefore is more widely applicable in comparison with the traditional thoracic cardiopulmonary resuscitation.

However, the abdominal device which drives cardiopulmonary resuscitation in a pull-push mode also has the following defects: The electrocardiographic changes of the patient can be monitored by only the pull-push information such as amplitude and displacement of the pull and push, the pull force and the push force displayed on the display, electrocardiographic changes of the patient cannot be accurately displayed; or an electrocardiograph monitor equipped with a display is required to be used in combination to perform the monitoring, but during the process of pulling and pushing the abdomen, the pull-push information on the display needs to be noted while the pull-push effect is required, which increases difficulties in operation and greatly reduces the effects of cardiopulmonary resuscitation. Limited by the golden time for rescuing patients suffering sudden heart arrest, efficiency is a main factor of the rescue. Besides, for rescue out of hospitals, the electrocardiographic changes of the patients cannot be monitored due to limit in devices used out of the hospitals, causing various inconvenience to rescue.

Thus it can be seen that the existing cardiopulmonary resuscitator are still inconvenient and defective in structure and in use, and need further improvement. How to create a portable cardiopulmonary resuscitator which can be conveniently used to rescue patients suffering from sudden heart arrest and improve the success rate of the rescue is an objective urgently to be solved.

### BRIEF SUMMARY OF THE INVENTION

The technical problem to be solved by the utility model is to provide a portable abdominal cardiopulmonary resuscitator which can conveniently rescue patients suffering sudden cardiac arrest and enhance the success rate of the rescue.

To solve the above technical problem, the utility model employs following technical solution:

A portable abdominal cardiopulmonary resuscitator for electrocardiogram monitoring includes an abdominal pull-push cardiopulmonary resuscitator body provided with a display and a central processing unit, wherein the abdominal push cardiopulmonary resuscitator body is also provided with an electrocardiographic electrode communication module which is connected with the central processing unit; the electrocardiographic electrode communication module is connected with electrocardiographic electrodes in a wireless or wired way; and the display is used for displaying abdominal pull-push information and electrocardiogram monitoring information.

Further, the portable abdominal cardiopulmonary resuscitator for electrocardiogram monitoring also includes electrocardiographic electrodes, and the electrocardiographic electrodes are connected with an electrocardiographic electrode communication module in a wireless or wired way.

Further, the wireless connection is blue-tooth connection.

Further, the abdominal pull-push cardiopulmonary resuscitator body adopts a vacuum adsorption structure, including a pressure plate and a vacuum adsorption cavity installed on the pressure plate; a cavity opening of the vacuum adsorption cavity is formed on the pressure plate; and the vacuum adsorption cavity is internally provided with soft grilles.

Further, the soft grilles are rubber grilles.

Further, the abdominal pull-push cardiopulmonary resuscitator body includes a pressure plate and a vacuum adsorption cavity installed on the pressure plate, and projections for pushing the abdominal artery are disposed on the bottom surface of the pressure plate.

Further, the bottom surface of the pressure plate is shaped as a pentagon; the pentagon is an axially symmetric structure; the projections for pushing the abdominal artery are positioned at axially symmetric corners of the pentagon.

Further, the abdominal pull-push cardiopulmonary resuscitator body also includes handles; two handles are provided, respectively positioned on the left and right sides of the display; and the handles are structured to have upwarped ends.

By employing the above technical solution, the utility model has at least the following advantages:

1. By adding the electrocardiographic monitoring function on the display and following the abdominal pull-push information on the display, medical staff or operators can observe the electrocardiographic changes of patients in real time during rescue without raising head to adjust the pull and push situations of the abdominal cardiopulmonary resuscitator in real time. The abdominal cardiopulmonary resuscitator is conveniently used, is not limited by the application sites, and greatly improves the success rate of rescue.

2. The wireless communication, for example blue-tooth communication, can further facilitate the operation.

3. The arrangement of the soft grilles in the vacuum adsorption cavity reduces the skin injuries of the patient caused by a large adsorption force and a concentrated adsorption face during vacuum adsorption.

4. The projections for pushing the abdominal artery is disposed on the bottom surface of the pressure plate to mainly press the abdominal artery, which can effectively improve coronary perfusion, namely delivering more fresh oxygen-enriched blood to the heart. This is a key for enhancing the success rate of the cardiopulmonary resuscitation.

5. The handles are structured to have upwarped ends, so operators can conveniently and easily apply force during operation.

### BRIEF DESCRIPTION OF THE DRAWINGS

Above is summary of the technical solution of the utility model. In order to better describe the technical means of the utility model, the utility model is described in further detail in conjunction with the attached drawings and the embodiments.

FIG. 1 is a top structural view of a portable abdominal cardiopulmonary resuscitator for electrocardiogram monitoring according to an embodiment.

FIG. 2 is a structural view of an electrocardiographic electrode.

FIG. 3 is a front structural view of a portable abdominal cardiopulmonary resuscitator for electrocardiogram monitoring according to an embodiment (partially sectional view of a vacuum adsorption cavity).

FIG. 4 is a bottom structural view of a pressure plate.

### DETAILED DESCRIPTION OF THE INVENTION

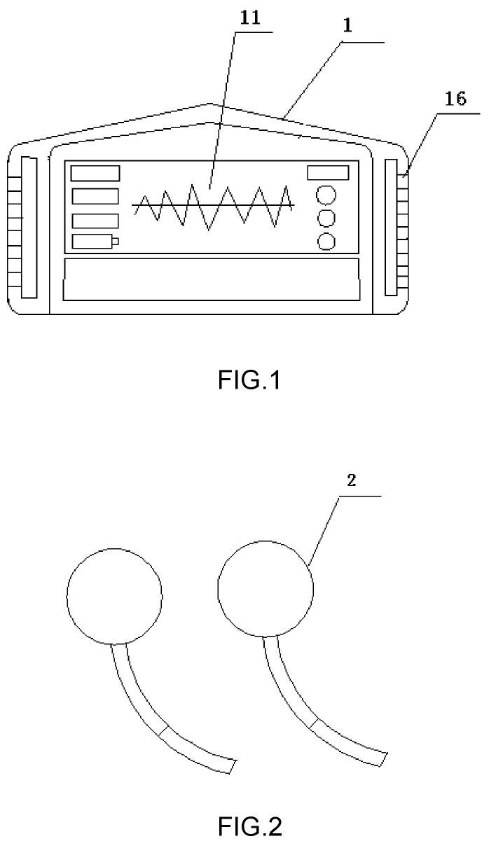

The utility model provides a portable abdominal cardiopulmonary resuscitator for electrocardiogram monitoring, as shown in FIG. 1, including an abdominal pull-push cardiopulmonary resuscitator body 1 provided with a display 11 and a central processing unit, wherein the abdominal pull-push cardiopulmonary resuscitator body 1 is also provided with an electrocardiographic electrode communication module which is connected with the central processing unit; the electrocardiographic electrode communication module is connected with electrocardiographic electrodes 2 as shown in FIG. 2 in a wireless or wired way; and the display 11 is used for displaying abdominal pull-push information and electrocardiogram monitoring information.

The resuscitator can be attached with the electrocardiographic electrodes before delivery or buyers can self-buy supporting electrocardiographic electrodes. For the wireless connection mode, blue-tooth connection is preferred.

In use, the electrocardiographic electrodes 2 are placed at corresponding positions on a human body. Following the instructions on the display of the abdominal pull-push cardiopulmonary resuscitator, medical staff or operators can observe the electrocardiographic changes of patients in real time during rescue without raising heads to adjust the pull and push situations of the abdominal cardiopulmonary resuscitator in real time. The abdominal cardiopulmonary resuscitator is conveniently used, is not limited by the application sites, and greatly improves the success rate of rescue.

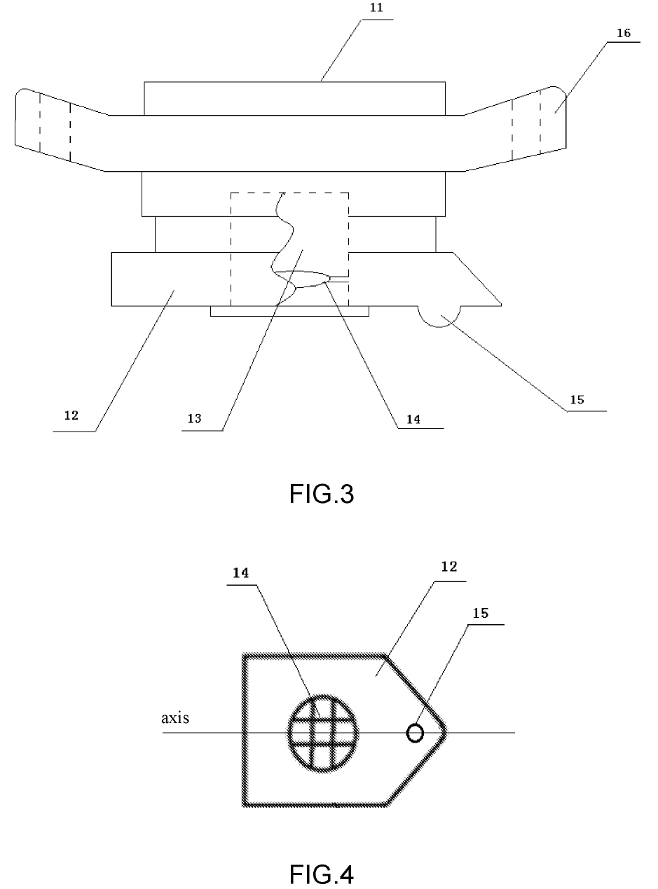

As shown in FIG. 3, the abdominal pull-push cardiopulmonary resuscitator body 1 adopts a vacuum adsorption structure, including a pressure plate 12 and a vacuum adsorption cavity 13 installed on the pressure plate 12; a cavity opening of the vacuum adsorption cavity 13 is formed on the pressure plate 12; and the vacuum adsorption cavity 13 is internally provided with soft grilles 14. Rubber grilles are preferred. The arrangement of the soft grilles 14 greatly reduces the skin injuries of the patient caused by a large adsorption force and a concentrated adsorption face during vacuum adsorption.

As shown in FIG. 3 and FIG. 4, projections 15 for pushing the abdominal artery are disposed on the bottom surface of the pressure plate 12. The bottom surface of the pressure plate 12 is shaped as a pentagon. The pentagon is an axially symmetric structure. The projections 15 for pushing the abdominal artery are positioned at axially symmetric corners of the pentagon.

In use, the pressure plate 12 of the abdominal pull-push cardiopulmonary resuscitator is placed at the middle-upper part of the abdomen of the patient, wherein the axially symmetric corners of the pentagon facing the chest, and the projections 15 for pushing the abdominal artery point at the abdominal artery. Then pull-push operation is carried out. During the process of implementing the abdominal pull-push cardiopulmonary resuscitation, the quick rise of the abdominal pressure can promote liquid in abdominal organs, which accounts for 25% of the blood supply of the human body, to flow back to the right atrium via veins of the lower cavity, and to maintain the aortic pressure continuously higher than the pressure in the right atrium, thus ensuring that the blood with a low oxygen content does not flow back to the heart. Pressing the abdominal artery with the projections 15 for pushing the abdominal artery can effectively improve coronary perfusion, namely delivering more fresh oxygen-enriched blood to the heart, which is a key for enhancing the success rate of the cardiopulmonary resuscitation. The abdominal pressing can also raise the diaphragm to drive the patient to breathe, and the pushing actions for attracting the abdominal wall in vacuum can descend the diaphragm to increase the negative pressure in the thoracic cavity, which aids flow of the air in the lung, thereby achieving effects in aspiration and circulation.

Besides, as shown in FIG. 1 and FIG. 3, the abdominal pull-push cardiopulmonary resuscitator body 1 also includes handles 16; two handles 16 are provided, respectively positioned on the left and right sides of the display 11; the handles 16 are structured to have upwarped ends, so operators can conveniently and easily apply force during pressing.

In use, a medical person or an operator can grip the handles 16, turn on the resuscitator, and after the resuscitator is adsorbed to the abdominal wall of the patient, pull and push the abdomen of the patient vertically. The recommended push stress is 50 kg±10%, and the recommended pull force is 30 kg±10%. In use, the display will display the electrocardiogram of the patient in real time and also display the pull-push information such as acting force, frequency, operation time length, adsorption state, etc. during the operation.

The utility model can improve the success rate of the rescue of patients, reduces the partial skin injuries for patients, and is applicable to be promoted.

The above is only a preferred embodiment of the utility model, and does not limit the utility model in any form. Those skilled in the field can make amendments, equivalent changes or modifications, which shall fall within the protective scope of the utility model, on the basis of the above technical solution.

What is claimed is:

1. A portable abdominal cardiopulmonary resuscitator for electrocardiogram monitoring comprises an abdominal pull-push cardiopulmonary resuscitator body provided with a display and a central processing unit, characterized in that the abdominal pull-push cardiopulmonary resuscitator body is also provided with an electrocardiographic electrode communication module which is connected with the central processing unit; the electrocardiographic electrode communication module is connected with electrocardiographic electrodes in a wireless or wired way; and the display is used for displaying abdominal pull-push information and electrocardiogram monitoring information.

2. The portable abdominal cardiopulmonary resuscitator for electrocardiogram monitoring according to claim 1, wherein the portable abdominal cardiopulmonary resuscitator for electrocardiogram monitoring also comprises electrocardiographic electrodes, and the electrocardiographic electrodes are connected with an electrocardiographic electrode communication module in a wireless or wired way.

3. The portable abdominal cardiopulmonary resuscitator for electrocardiogram monitoring according to claim 1, wherein the wireless connection is blue-tooth connection.

4. The portable abdominal cardiopulmonary resuscitator for electrocardiogram monitoring according to claim 2, wherein the wireless connection is blue-tooth connection.

5. The portable abdominal cardiopulmonary resuscitator for electrocardiogram monitoring according to claim 1, wherein the abdominal pull-push cardiopulmonary resuscitator body adopts a vacuum adsorption structure, including a pressure plate and a vacuum adsorption cavity installed on the pressure plate; a cavity opening of the vacuum adsorption cavity is formed on the pressure plate; the vacuum adsorption cavity is internally provided with soft grilles.

6. The portable abdominal cardiopulmonary resuscitator for electrocardiogram monitoring according to claim 2, wherein the abdominal pull-push cardiopulmonary resuscitator body adopts a vacuum adsorption structure, including a pressure plate and a vacuum adsorption cavity installed on the pressure plate; a cavity opening of the vacuum adsorption cavity is formed on the pressure plate; the vacuum adsorption cavity is internally provided with soft grilles.

7. The portable abdominal cardiopulmonary resuscitator for electrocardiogram monitoring according to claim 5, wherein the soft grilles are rubber grilles.

8. The portable abdominal cardiopulmonary resuscitator for electrocardiogram monitoring according to claim 6, wherein the soft grilles are rubber grilles.

9. The portable abdominal cardiopulmonary resuscitator for electrocardiogram monitoring according to claim 1, wherein the abdominal pull-push cardiopulmonary resuscitator body includes a pressure plate and a vacuum adsorption cavity installed on the pressure plate, and projections for pushing the abdominal artery are disposed on the bottom surface of the pressure plate.

10. The portable abdominal cardiopulmonary resuscitator for electrocardiogram monitoring according to claim 2, wherein the abdominal pull-push cardiopulmonary resuscitator body includes a pressure plate and a vacuum adsorption cavity installed on the pressure plate, and projections for pushing the abdominal artery are disposed on the bottom surface of the pressure plate.

11. The portable abdominal cardiopulmonary resuscitator for electrocardiogram monitoring according to claim 9, wherein the bottom surface of the pressure plate is shaped as a pentagon; the pentagon is an axially symmetric structure; the projections for pushing the abdominal artery are positioned at axially symmetric corners of the pentagon.

12. The portable abdominal cardiopulmonary resuscitator for electrocardiogram monitoring according to claim 10, wherein the bottom surface of the pressure plate is shaped as a pentagon; the pentagon is an axially symmetric structure; the projections for pushing the abdominal artery are positioned at axially symmetric corners of the pentagon.

13. The portable abdominal cardiopulmonary resuscitator for electrocardiogram monitoring according to claim 1, wherein the abdominal pull-push cardiopulmonary resuscitator body also includes handles; two handles are provided, respectively positioned on the left and right sides of the display; and the handles are structured to have upwarped ends.

14. The portable abdominal cardiopulmonary resuscitator for electrocardiogram monitoring according to claim 2, wherein the abdominal pull-push cardiopulmonary resuscitator body also includes handles; two handles are provided, respectively positioned on the left and right sides of the display; and the handles are structured to have upwarped ends.
